(19) United States
(12) Patent Application Publication
(10) Pub. No.: US 2007/0135401 A1
(43) Pub. Date: Jun. 14, 2007

(54) Lestaurtinib Crystalline Form 1, crystalline lestaurimib anhydrate and amorphous lestaurimib

(76) Inventors: Walter Dziki, Libertyville, IL (US); Gowdahalli N. Subbarao, Mundelein, IL (US); Rodger F. Henry, Wildwood, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/636,188

(22) Filed: Dec. 8, 2006

Related U.S. Application Data
(60) Provisional application No. 60/748,855, filed on Dec. 9, 2005.

(51) Int. Cl. A61K 31/407; C07D 487/14

(52) U.S. Cl. 514/183; 540/460

(57) ABSTRACT

Lestaurtinib Crystalline Form 1, isolated crystalline lestaurtinib anhydrate and amorphous lestaurtinib, processes to reproducibly make them and methods of treating patients using them.

This application claims priority to U.S. Provisional Application Ser. No. 60/748,855, filed Dec. 9, 2005.

### FIELD OF THE INVENTION

This invention pertains to Lestaurtinib Crystalline Form 1, isolated crystalline lestaurtinib anhydrate and amorphous lestaurtinib, processes to reproducibly make them and methods of treating patients using them.

### BACKGROUND OF THE INVENTION

Lestaurtinib is an semi-synthetic, orally bioavailable receptor-tyrosine kinase inhibitor that has been shown to have therapeutic utility in treating diseases such as acute myeloid leukemia, chronic myeloid leukemia and acute lymphocytic leukemia. It is a synthetic derivative of K-252a, a fermentation product of Nonomurea longicatena, and belongs to a class of indolocarbazole alkaloids. U.S. Pat. No. 4,923,986 describes lestaurtinib, also known as (9S-(9α,10β,12α))-2,3,9,10,11,12-hexahydro-10-hydroxy-10-(hydroxymethyl)-9-methyl-9,12-epoxy-1H-diindolo[1,2,3-fg:3′,2′,1′-kl]pyrrolo[3,4-i][1,6]benzodiazocin-1-one (CAS Registry No. 111358-88-4) and utility thereof.

Different crystalline forms lestaurtinib can have different melting points, solubilities or rates of solubility, which physical properties, either alone or in combination, can effect its bioavailibility. Because knowledge of crystallinity, or lack thereof, of lestaurtinib can provide guidance during clinical development, there is an existing need for identification of different crystalline forms of lestaurtinib, processes to reproducibly make them and methods of treating patients using them.

### SUMMARY OF THE INVENTION

One embodiment of this invention, therefore, pertains to Lestaurtinib Crystalline Form 1 characterized, when measured at about 25° C. with Cu—Kα radiation, by a powder diffraction pattern with at least three peaks having respective 2θ values of about 6.8°, 8.5°, 9.7°, 12.0°, 13.2°, 14.2°, 14.7°, 15.0°, 15.5°, 16.9°, 17.5°, 17.9°, 19.3°, 20.0°, 20.4°, 25.1°, 25.6°, 25.8°, 26.3° or 26.6°.

Another embodiment pertains to Lestaurtinib Crystalline Form 1 characterized in the triclinic crystal system and P1 space group, when measured at about −100° C. with Mo—Kα radiation, by lattice parameters a, b and c of 11.235 Å±0.002 Å, 13.317 Å±0.004 Å, 7.095 Å±0.003 Å, respectively, and α, β and γ of 92.33°±0.03°, 107.78°±0.02° and 100.95°±0.02°, respectively.

Another embodiment pertains to compositions comprising or made from Lestaurtinib Crystalline Form 1 and an excipient.

Still another embodiment pertains to a method of treating patients having a disease caused or exascerbated by unregulated or overexpressed receptor-tyrosine kinase comprising administering thereto a therapeutically acceptable amount of Lestaurtinib Crystalline Form 1.

Still another embodiment pertains to a method of treating patients having acute myeloid leukemia comprising administering thereto a therapeutically acceptable amount of Lestaurtinib Crystalline Form 1.

Still another embodiment pertains to a method of treating patients having chronic myeloid leukemia comprising administering thereto a therapeutically acceptable amount of Lestaurtinib Crystalline Form 1.

Still another embodiment pertains to a method of treating patients having acute lymphocytic leukemia comprising administering thereto a therapeutically acceptable amount of Lestaurtinib Crystalline Form 1.

Still another embodiment pertains to a method of treating patients having chronic lymphocytic leukemia comprising administering thereto a therapeutically acceptable amount of Lestaurtinib Crystalline Form 1.

Still another embodiment pertains to a process for making Lestaurtinib Crystalline Form 1 comprising:

making lestaurtinib, or a solvate thereof, and isolating or not isolating the lestaurtinib, or the solvate thereof;

providing a mixture comprising the lestaurtinib, or the solvate thereof, and solvent, wherein the lestaurtinib, or the solvate thereof, is completely dissolved in the solvent;

causing Lestaurtinib Crystalline Form 1 to exist in the mixture, the Lestaurtinib Crystalline Form 1, when isolated and characterized the triclinic crystal system and P1 space group at about −100° C. with Mo—Kα radiation, by lattice parameters a, b and c of 11.235 Å±0.002 Å, 13.317 Å±0.004 Å, 7.095 Å±0.003 ↑1, respectively, and α, β and γ of 92.33°±0.03°, 107.78°±0.02° and 100.95°±0.02°, respectively; and

isolating the Lestaurtinib Crystalline Form 1.

Still another embodiment pertains to a process for making Lestaurtinib Crystalline Form 1 comprising:

making lestaurtinib, or a solvate thereof, and isolating or not isolating the lestaurtinib, or the solvate thereof;

providing a mixture comprising the lestaurtinib, or the solvate thereof, and solvent, wherein the lestaurtinib is completely dissolved in the solvent and the solvent is supersaturated with the lestaurtinib;

causing Lestaurtinib Crystalline Form 1 to exist in the mixture, the Lestaurtinib Crystalline Form 1, when isolated and characterized the triclinic crystal system and P1 space group at about −100° C. with Mo—Kα radiation, by lattice parameters a, b and c of 11.235 Å±0.002 Å, 13.317 Å±0.004 Å, 7.095 Å±0.003 Å, respectively, and α, β and γ of 92.33°±0.03°, 107.78°±0.02° and 100.95°±0.02°, respectively; and

isolating the Lestaurtinib Crystalline Form 1.

Still another embodiment pertains to a process for making Lestaurtinib Crystalline Form 1 comprising:

making and isolating or not isolating lestaurtinib or a solvate thereof;

providing a mixture comprising the lestaurtinib and solvent, wherein the lestaurtinib is partially soluble in the solvent;

allowing Lestaurtinib Crystalline Form 1 to develop in the mixture; and

isolating the Lestaurtinib Crystalline Form 1.

Still another embodiment pertains to a process for making Lestaurtinib Crystalline Form 1 comprising:

making and isolating or not isolating lestaurtinib or a solvate thereof;

providing a mixture comprising the lestaurtinib and ethanol, in which the lestaurtinib is partially soluble in the ethanol;

allowing Lestaurtinib Crystalline Form 1 to develop in the mixture; and

isolating the Lestaurtinib Crystalline Form 1.

Still another embodiment pertains to a process for making Lestaurtinib Crystalline Form 1 comprising:

providing a mixture comprising lestaurtinib and ethanol at about 0° C. to about 33° C., wherein the lestaurtinib is partially soluble in the ethanol;

allowing Lestaurtinib Crystalline Form 1 to develop in the mixture; and

isolating the Lestaurtinib Crystalline Form 1.

Still another embodiment pertains to isolated crystalline lestaurtinib anhydrate.

Still another embodiment pertains to compositions comprising or made from isolated crystalline lestaurtinib anhydrate and an excipient.

Still another embodiment pertains to treating patients having a disease caused or exascerbated by unregulated or overexpressed receptor-tyrosine kinase comprising administering thereto a therapeutically acceptable amount of isolated crystalline lestaurtinib anhydrate.

Still another embodiment pertains to a method of treating patients having acute myeloid leukemia comprising administering thereto a therapeutically acceptable amount of isolated crystalline lestaurtinib anhydrate.

Still another embodiment pertains to a method of treating patients having chronic myeloid leukemia comprising administering thereto a therapeutically acceptable amount of isolated crystalline lestaurtinib anhydrate.

Still another embodiment pertains to a method of treating patients having acute lymphocytic leukemia comprising administering thereto a therapeutically acceptable amount of isolated crystalline lestaurtinib anhydrate.

Still another embodiment pertains to a method of treating patients having chronic lymphocytic leukemia comprising administering thereto a therapeutically acceptable amount of isolated crystalline lestaurtinib anhydrate.

Still another embodiment pertains to a process for making isolated crystalline lestaurtinib anhydrate comprising:

making a crystalline lestaurtinib hydrate or a mixture of crystalline lestaurtinib hydrates;

removing water from the crystalline lestaurtinib hydrate or the mixture of crystalline lestaurtinib hydrates; and

isolating the crystalline lestaurtinib anhydrate.

Still another embodiment pertains to a process for making isolated crystalline lestaurtinib anhydrate comprising:

making a crystalline lestaurtinib hydrate or a mixture of crystalline lestaurtinib hydrates;

heating the crystalline lestaurtinib hydrate or the mixture of crystalline lestaurtinib hydrates above about 40° C., with or without a dessicant and under a vacuum of less than 760 mm Hg or at about 760 mm Hg; and

isolating the crystalline lestaurtinib anhydrate.

Still another embodiment pertains to a process for making isolated crystalline lestaurtinib anhydrate comprising:

making a crystalline lestaurtinib hydrate or a mixture of crystalline lestaurtinib hydrates;

heating the crystalline lestaurtinib hydrate or the mixture of crystalline lestaurtinib hydrates between about 80° C. and 100° C., with or without a dessicant and under a vacuum of less than 760 mm Hg or at about 760 mm Hg; and

isolating the crystalline lestaurtinib anhydrate.

Still another embodiment pretains to a process for making Lestaurtinib Crystalline Form 1 comprising treatment of a carboxyl-protected intermediate in said process with a reducing agent and subsequently crystallizing or recrystallizing said lestaurtinib to said Lestaurtinib Crystalline Form 1, wherein said process comprises direct crystallization of Lestaurtinib Crystalline Form 1 from a solid, semisolid or syrup having therewith one or more than one solvent from said carboxylic acid-protected intermediate reduction.

Still another embodiment pretains to a process for making Lestaurtinib Crystalline Form 1 comprising treatment of a carboxyl-protected intermediate in said process with a reducing agent and subsequently crystallizing or recrystallizing said lestaurtinib to said Lestaurtinib Crystalline Form 1, wherein said process comprises direct crystallization of Lestaurtinib Crystalline Form 1 from a solid having therewith one or more than one solvent from the group consisting of water, tetrahydrofuran, toluene, methanol and ethanol from said carboxylic acid deprotection reaction.

Still another embodiment pretains to a process for making Lestaurtinib Crystalline Form 1 comprising treatment of a carboxyl-protected intermediate in said process with a lithium aluminum hydride or sodium borohydride and subsequently crystallizing or recrystallizing said lestaurtinib to said Lestaurtinib Crystalline Form 1, wherein said process comprises direct crystallization of Lestaurtinib Crystalline Form 1 from a solid having therewith one or more than one solvent from the group consisting of water, tetrahydrofuran, toluene, methanol and ethanol from said carboxylic acid deprotection reaction.

Still another embodiment pretains to a process for making Lestaurtinib Crystalline Form 1 comprising treatment of methyl(9S-(9α,10β,12α))-2,3,9,10,11,12-hexahydro-10-hydroxy-10-(carboxylate)-9-methyl-9,12-epoxy-1H-diindolo[1,2,3-fg:3′,2′,1′-kl]pyrrolo[3,4-i][1,6]benzodiazocin-1-one with a reducing agent and subsequently crystallizing or recrystallizing said lestaurtinib to said Lestaurtinib Crystalline Form 1, wherein said process comprises direct crystallization of Lestaurtinib Crystalline Form 1 from a solid, semisolid or syrup having therewith one or more than one solvent from said carboxylic acid-protected intermediate reduction.

Still another embodiment pretains to a process for making Lestaurtinib Crystalline Form 1 comprising treatment of methyl(9S-(9α,10β,12α))-2,3,9,10,11,12-hexahydro-10-hydroxy-10-(carboxylate)-9-methyl-9,12-epoxy-1H-diindolo[1,2,3-fg:3′,2′,1′-kl]pyrrolo[3,4-i][1,6]benzodiazocin-1-one with a reducing agent and subsequently crystallizing or recrystallizing said lestaurtinib to said Lestaurtinib Crystalline Form 1, wherein said process comprises direct crystallization of Lestaurtinib Crystalline Form 1 from a solid having therewith one or more than one solvent from the group consisting of water, tetrahydrofuran, toluene, methanol and ethanol from said carboxylic acid deprotection reaction.

Still another embodiment pretains to a process for making Lestaurtinib Crystalline Form 1 comprising treatment of methyl(9S-(9α,10β,12α))-2,3,9,10,11,12-hexahydro-10-hydroxy-10-(carboxylate)-9-methyl-9,12-epoxy-1H-diindolo[1,2,3-fg:3′,2′,1′-kl]pyrrolo[3,4-i][1,6]benzodiazocin-1-one with a lithium aluminum hydride or sodium borohydride and subsequently crystallizing or recrystallizing said lestaurtinib to said Lestaurtinib Crystalline Form 1, wherein said process comprises direct crystallization of Lestaurtinib Crystalline Form 1 from a solid having therewith one or more than one solvent from the group consisting of water, tetrahydrofuran, toluene, methanol and ethanol from said carboxylic acid deprotection reaction.

Still another embodiment pretains to a process for making Lestaurtinib Crystalline Form 1 comprising treatment of (9S-(9α,10β,12α))-2,3,9,10,11,12-hexahydro-10-hydroxy-10-(carboxy)-9-methyl-9,12-epoxy-1H-diindolo[1,2,3-fg:3′,2′,1′-kl]pyrrolo[3,4-i][1,6]benzodiazocin-1-one with a reducing agent and subsequently crystallizing or recrystallizing said lestaurtinib to said Lestaurtinib Crystalline Form 1, wherein said process comprises direct crystallization of Lestaurtinib Crystalline Form 1 from a solid, semisolid or syrup having therewith one or more than one solvent from said carboxylic acid-protected intermediate reduction.

Still another embodiment pretains to a process for making Lestaurtinib Crystalline Form 1 comprising treatment of (9S-(9α, 10β, 12α))-2,3,9,10,11,12-hexahydro-10-hydroxy-10-(carboxy)-9-methyl-9,12-epoxy-1H-diindolo[1,2,3-fg:3′,2′,1′-kl]pyrrolo[3,4-i][1,6]benzodiazocin-1-one with a reducing agent and subsequently crystallizing or recrystallizing said lestaurtinib to said Lestaurtinib Crystalline Form 1, wherein said process comprises direct crystallization of Lestaurtinib from a solid having therewith one or more than one solvent from the group consisting of water, tetrahydrofuran, toluene, methanol and ethanol from said carboxylic acid deprotection reaction.

Still another embodiment pretains to a process for making Lestaurtinib Crystalline Form 1 comprising treatment of (9S-(9α,10β,12α))-2,3,9,10,11,12-hexahydro-10-hydroxy-10-(carboxy)-9-methyl-9,12-epoxy-1H-diindolo[1,2,3-fg:3′,2′,1′-kl]pyrrolo[3,4-i][1,6]benzodiazocin-1-one with a lithium aluminum hydride or sodium borohydride and subsequently crystallizing or recrystallizing said lestaurtinib to said Lestaurtinib Crystalline Form 1, wherein said process comprises direct crystallization of Lestaurtinib Crystalline Form 1 from a solid having therewith one or more than one solvent from the group consisting of water, tetrahydrofuran, toluene, methanol and ethanol from said carboxylic acid deprotection reaction.

Still another embodiment pertains to amorphous lestaurtinib.

Still another embodiment pertains to compositions comprising or made from amorphous lestaurtinib and an excipient.

Still another embodiment pertains to treating patients having a disease caused or exascerbated by unregulated or overexpressed receptor-tyrosine kinase comprising administering thereto a therapeutically acceptable amount of amorphous lestaurtinib.

Still another embodiment pertains to a method of treating patients having acute myeloid leukemia comprising administering thereto a therapeutically acceptable amount of amorphous lestaurtinib.

Still another embodiment pertains to a method of treating patients having chronic myeloid leukemia comprising administering thereto a therapeutically acceptable amount of amorphous lestaurtinib.

Still another embodiment pertains to a method of treating patients having acute lymphocytic leukemia comprising administering thereto a therapeutically acceptable amount of amorphous lestaurtinib.

Still another embodiment pertains to a method of treating patients having chronic lymphocytic leukemia comprising administering thereto a therapeutically acceptable amount of amorphous lestaurtinib.

### DETAILED DESCRIPTION OF THE INVENTION

Different crystalline forms of a given drug have physical, pharmaceutical, physiological and biological properties which can sharply differ from one other. This invention pertains to crystalline forms of lestaurtinib and amorphous lestaurtinib. It is meant to be understood that the term “lestaurtinib,” as used herein, without a designation of crystallinity or lack thereof, means a particular crystalline or amorphous form of lestaurtinib, lestaurtinib in solution or a mixture thereof.

Lestaurtinib Crystalline Form 1 is the most thermodynamically stable crystalline form of lestaurtinib at ambient temperature (about 25° C.). It is a non-hygroscopic, crystalline form having less than 0.05% moisture, as determined by thermogravimetric analysis (TGA). Additionally, Form I is not known to undergo solid state conversion regardless of temperature or relative humidity (RH).

Crystalline lestaurtinib trihydrate is stable at about 45% to about 95% RH at ambient temperature.

Crystalline lestaurtinib monohydrate is stable at about 10% to about 40% relative RH at about 25° C. At ambient temperature and above 40% RH, the monohydrate readily converts to the trihydrate. When ground with a mortar and pestle, crystalline lestaurtinib monohydrate's ability to absorb water is reduced by a factor of about 6. Thus it takes about 6 times longer to absorb similar amounts of water when ground than unground.

Lestaurtinib monohydrate can be made by exposing the trihydrate to RH levels of 40% or less at ambient temperature or by heating the trihydrate between 80° C. and 200° C., followed by exposure to ambient conditions for about 10 minutes. After the exposure period, the sample must be stored in a sealed container.

Crystalline lestaurtinib anhydrate is stable at ambient temperature between about 0% and about 5% RH but absorbs moisture above 5% RH to form crystalline lestaurtinib monohydrate. Existence of crystalline lestaurtinib anhydrate was demonstrated by dynamic moisture sorption gravimetry (DMSG) which displayed, at 25° C., a solid-state phase between 0% and 5% RH with less than 0.5% water. Because moisture-mediated crystallization was not observed during RH levels between 5% and 10%, it was concluded that the solid at 5% RH was crystalline; and because the solid contained less than 0.5% water, it was also determined that it was an anhydrate.

Crystalline lestaurtinib anhydrate can be produced by either exposing crystalline lestaurtinib anhydrate to RH levels 5% or less at ambient temperature or by heating the trihydrate between 80° C. and 200° C. and storing the product under moisture-free conditions. The sample can absorb water from the atmosphere during the transfer period.

Crystalline lestaurtinib hemihydrate hemiacetonitrileate is a crystalline mixed solvate with about ½ mole equivalent of water and about ½ mole equivalent of acetonitrile. The solvents are entrapped within the crystal lattice and can be removed by heating a sample between 130° C. and 220° C.

Powder X-Ray diffraction (PXRD) pdata were obtained with a Scintag model X1 unit with a copper target (1.54060 Å wavelength radiation: 45 Kv and 40 ma); scan rate: 1° per minute continuous; and a scan range of 2-40° 2θ at ambient temperature using a Peltier cooled detector tuned for copper radiation. All XRPD samples were gently ground to a fine powder in a mortar and pestle prior to analysis.

Differential scanning calorimetry (DSC) data were obtained using a TA Instruments Model 3100 Thermal Analyst with a Model 2910 DSC Module. Samples were prepared in uncrimped closed aluminum pans. Typical DSC sample weights were about 1-4 mg. Thermograms were acquired at a heating rate of 5° C. per minute under nitrogen purge with a flow rate of about 40 mL per minute. Thermograms for Lestaurtinib Crystalline Form 1 and amorphous lestaurtinib were obtained between room temperature and about 330° C. Lestaurtinib Crystalline Form 1 exhibited an endotherm with onset at 268.2° C. and peak temperature at 277.4° C. Amorphous lestaurtinib exhibited a broad endotherm with onset at 26.6° C. and peak temperature of 57.0° C.

TGA data were obtained using a TA Instruments Model 3100 and 5200 Thermal Analysts with a Model 2950 Hi Res TGA module. TGA thermograms were acquired at a heating rate of 5° C. per minute, under nitrogen purge with a flow rate of about 40 mL per minute. Lestaurtinib Crystalline Form 1 exhibited 0.04% weight loss up to about 100° C. Amorphous lestaurtinib exhibited 5.56% weight loss up to about 184° C.

Dynamic moisture sorption gravimetry (DMSG) (hygroscopicity) data were obtained on a VTI Corporation model MB 300 G sorption microbalance using vacuum to control RH. The automated system controlled the RH and temperature to which each sample was exposed, while continuously recording sample weight changes. Sorption and desorption isotherms were performed at 25±0.1° C. with 5±1% RH step intervals from 0-95% RH. Samples of about 15-30 mg were dried under vacuum for up to 3 hours (approximate RH =0-1% RH) before each experiment. The weight loss observed during the drying period was used to estimate how tightly each sample held onto water. After the drying period, sorption isotherms started at 5% RH. A critical weight equilibrium of less than 5 mg weight change over three 5 minutes periods was used to move to the next step. When the equilibrium conditions were achieved for the 95% RH step, the desorption isotherm was started. Lestaurtinib Crystalline Form 1 exhibited moisture uptake up to about 0.4% with an equilibrium moisture content (EMC or mole water per mole drug) of about 0.1 and a nominal moisture uptake of about 0.2% (w/w) with an EMC of about 0.05. Meaning that any water present in the sample was surface water and not lattice water.

Solubility of Lestaurtinib Crystalline Form 1 and anhydrous lestaurtinib at 26° C. and 2-8° C. was determined using HPLC. Saturated solutions of the solid-state phase in ethanol (200 poof) were obtained by slurrying a sample in ethanol for two days at 26° C. and at 2-8° C. After slurrying, each solution was filtered through 0.45 μm micron filter and the level of CEP-701 was determined by HPLC. The solubility data is presented in TABLE 1.

TABLE 1
Solubility of Lestaurtinib Crystalline Form 1 and
Anhydrous Lestaurtinib In 200 Proof Ethanol

Solubility (mg/mL)

| Form | (2-8° C.) | (26° C.) |
|---|---|---|
| Form I | 2.38 | 2.60 |
| Amorphous | 22.1 | 4.05 |

These data demonstrate differences in one particular physical property (i.e. solubility) of lestaurtinib.

To determine the most stable crystalline form of lestaurtinib, forms of lestaurtinib were slurried with 200 proof ethanol for 48 hours at 26° C. and at 2-8° C. The forms of lestaurtinib remaining were analyzed by PXRD to determine if any crystal form conversion had occurred. Results, in Table 2, show that each form of lestaurtinib tested converted to Lestaurtinib Crystalline Form I, indicating that Lestaurtinib Crystalline Form 1 is the most stable crystalline form of lestaurtinib at ambient temperature and at 2-8° C.

TABLE 2
Crystalline Form Of Lestaurtinib After Contact
with 200 Proof Ethanol For 48 Hours

| | Form After Mixing With Ethanol | |
|---|---|---|
| | For 48 Hours | |
| Form Before Mixing With Ethanol | 2-8° C. | 26° C. |
| 1 | 1 | 1 |
| hydrated | 1 | 1 |
| hemi: hydrate/acetonitrileate | 1 | 1 |
| amorphous | 1 | 1 |

The term “amorphous,” as used herein, means a supercooled liquid or a viscous liquid which looks like a solid but does not have a regularly repeating arrangement of molecules that is maintained over a long range and does not have a melting point but rather softens or flows above its glass transition temperature.

The term “anti-solvent,” as used herein, means a solvent in which a compound is substantially insoluble.

The term “Lestaurtinib Crystalline Form 1,” as used herein, means the most thermodynamically stable crystalline form of lestaurtinib at 25° C.

The term “crystalline,” as used herein, means having a regularly repeating arrangement of molecules or external face planes.

The term “isolating” as used herein, means separating a compound from a solvent, anti-solvent, or a mixture of solvent and anti-solvent to provide a solid, semisolid or syrup. This is typically accomplished by means such as centrifugation, filtration with or without vacuum, filtration under positive pressure, distillation, evaporation or a combination thereof. Isolating may or may not be accompanied by purifying during which the chemical, chiral or chemical and chiral purity of the isolate is increased. Purifying is typically conducted by means such as crystallization, distillation, extraction, filtration through acidic, basic or neutral alumina, filtration through acidic, basic or neutral charcoal, column chromatography on a column packed with a chiral stationary phase, filtration through a porous paper, plastic or glass barrier, column chromatography on silica gel, ion exchange chromatography, recrystallization, normal-phase high performance liquid chromatography, reverse-phase high performance liquid chromatography, trituration and the like.

The term “miscible,” as used herein, means capable of combining without separation of phases.

The term “solvate,” as used herein, means having on a surface, in a lattice or on a surface and in a lattice, a solvent such as water, acetic acid, acetone, acetonitrile, benzene, chloroform, carbon tetrachloride, dichloromethane, dimethylsulfoxide, 1,4-dioxane, ethanol, ethyl acetate, butanol, tert-butanol, N,N-dimethylacetamide, N,N-dimethylformamide, formamide, formic acid, heptane, hexane, isopropanol, methanol, methyl ethyl ketone, 1-methyl-2-pyrrolidinone, mesitylene, nitromethane, polyethylene glycol, propanol, 2-propanone, pyridine, tetrahydrofuran, toluene, xylene, mixtures thereof and the like. A specific example of a solvate is a hydrate, wherein the solvent on the surface, in the lattice or on the surface and in the lattice, is water. Hydrates may or may not have solvents other than water on the surface, in the lattice or on the surface and in the lattice of a substance.

The term “solvent,” as used herein, means a substance, typically a liquid, that is capable of completely or partially dissolving another substance, typically a solid. Solvents for the practice of this invention include water, acetic acid, acetone, acetonitrile, benzene, chloroform, carbon tetrachloride, dichloromethane, dimethylsulfoxide, 1,4-dioxane, ethanol, ethyl acetate, butanol, tert-butanol, N,N-dimethylacetamide, N,N-dimethylformamide, formamide, formic acid, heptane, hexane, isopropanol, methanol, methyl ethyl ketone, 1-methyl-2-pyrrolidinone, mesitylene, nitromethane, polyethylene glycol, propanol, 2-propanone, pyridine, tetrahydrofuran, toluene, xylene, mixtures thereof and the like.

The term “supersaturated,” as used herein, means having a compound in a solvent in which it is completely dissolved at a certain temperature but at which the solubility of the compound in the solvent at that certain temperature is exceeded.

Unless stated otherwise, percentages stated throughout this specification are weight/weight (w/w) percentages.

Mixtures comprising lestaurtinib and solvent may or may not have chemical and diastereomeric impurities, which, if present, may be completely soluble, partially soluble or essentially insoluble in the solvent. The level of chemical or diastereomeric impurity in the mixture may be lowered before or during isolation of Lestaurtinib Crystalline Form 1 by means such as distillation, extraction, filtration through acidic, basic or neutral alumina, filtration through acidic, basic or neutral charcoal, column chromatography on a column packed with a chiral stationary phase, filtration through a porous paper, plastic or glass barrier, column chromatography on silica gel, ion exchange chromatography, recrystallization, normal-phase high performance liquid chromatography, reverse-phase high performance liquid chromatography, trituration and the like.

Causing Lestaurtinib Crystalline Form 1 to exist in a mixture comprising lestaurtinib and solvent, wherein the lestaurtinib is completely dissolved in the solvent, is nucleation. In a preferred embodiment for the practice of this invention, nucleation of Lestaurtinib Crystalline Form 1 is made to occur in a solvent which is supersaturated with of the lestaurtinib.

Mixtures of lestaurtinib and solvent, wherein the lestaurtinib is completely dissolved in the solvent may be prepared from a crystalline lestaurtinib, amorphous lestaurtinib or a mixture thereof.

For the practice of this invention, nucleation may be made to occur in a solution by techniques that are well-known to those skilled in the art such as solvent removal, temperature change, solvent-miscible anti-solvent addition, solvent-immiscible anti-solvent addition, seed crystal addition of Lestaurtinib Crystalline Form 1, chafing or scratching the interior of the container, preferably a glass container with a glass rod or a glass bead or beads, or by a combination thereof.

It is meant to be understood that, because many solvents and anti-solvents contain impurities, the level of impurities in solvents and anti-solvents for the practice of this invention, if present, are at a low enough concentration that they do not interfere with the intended use of the solvent in which they are present. Solvents used were HPLC, reagent or USP grade and were used as received.

The term “C₁-alkyl,” as used herein, means methyl.

The term “C₂-alkyl,” as used herein, means ethyl.

The term “C₃-alkyl,” as used herein, means prop-1-yl and prop-2-yl(isopropyl).

The term “C₄-alkyl,” as used herein, means but-1-yl, but-2-yl, 2-methylprop-1-yl and 2-methylprop-2-yl(tert-butyl).

The term “C₅-alkyl,” as used herein, means 2,2-dimethylprop-1-yl(neo-pentyl), 2-methylbut-1-yl, 2-methylbut-2-yl, 3-methylbut-1-yl, 3-methylbut-2-yl, pent-1-yl, pent-2-yl and pent-3-yl.

The term “C₆-alkyl,” as used herein, means 2,2-dimethylbut-1-yl, 2,3-dimethylbut-1-yl, 2,3-dimethylbut-2-yl, 3,3-dimethylbut-1-yl, 3,3-dimethylbut-2-yl, 2-ethylbut-1-yl, hex-1-yl, hex-2-yl, hex-3-yl, 2-methylpent-1-yl, 2-methylpent-2-yl, 2-methylpent-3-yl, 3-methylpent-1-yl, 3-methylpent-2-yl, 3-methylpent-3-yl, 4-methylpent-1-yl and 4-methylpent-2-yl.

The term “carboxyl-protected intermediate,” as used herein means an intermediate having a C(O)OH carboxyl moiety to which is attached a carboxyl protecting group.

The term “carboxyl protecting group,” as used herein means any moiety that can be attached to a C(O)OH moiety to make it less succeptable to undesired reaction during synthesis. Specific examples of carboxyl protecting groups include, bur are not limited to, phenyl, naphthyl, furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thiophenyl, triazinyl, 1,2,3-triazolyl acetoxymethyl, allyl, benzoylmethyl, benzyloxymethyl, tert-butyldiphenylsilyl, diphenylmethyl, cyclobutyl, cyclohexyl, cyclopentyl, cyclopropyl, diphenylmethylsilyl, para-methoxybenzyl, methoxymethyl, methoxyethoxymethyl, methylthiomethyl, para-nitrobenzyl, phenyl, 2,2,2-trichloroethyl, triethylsilyl, 2-(trimethylsilyl)ethyl, 2-(trimethylsilyl)ethoxymethyl, triphenylmethyl or C₁-alkyl, C₂-alkyl, C₃-alkyl, C₄-alkyl, C₅-alkyl or C₆-alkyl, each of which is unsubstituted or substituted with phenyl, naphthyl, furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thiophenyl, triazinyl, 1,2,3-triazolyl and the like.

The invention provides methods of treating diseases and conditions in a patient comprising administering thereto a therapeutically effective amount of lestaurtinib. Accordingly, lestaurtinib is useful for treating a variety of therapeutic indications. For example, lestaurtinib is useful for the treatment of cancers such as carcinomas of the pancreas, prostate, breast, thyroid, colon and lung; malignant melanomas; glioblastomas; neuroectodermal-derived tumors including Wilm's tumor, neuroblastomas and medulloblastomas; and leukemias such as acute myeloid leukemia (AML), chronic myeloid leukemia (CML), acute lymphocytic leukemia (ALL), chronic lymphocytic leukemia (CLL); pathological conditions of the prostate such as prostatic hypertrophy or prostate cancer; carcinomas of the pancreas, such as pancreatic ductal adenocarcinoma (PDAC); hyperproliferative disorders such as proliferative skin disorders including actinic keratosis, basal cell carcinoma, squamous cell carcinoma, fibrous histiocytoma, dermatofibrosarcoma protuberans, hemangioma, nevus flammeus, xanthoma, Kaposi's sarcoma, mastocytosis, mycosis fungoides, lentigo, nevocellular nevus, lentigo maligna, malignant melanoma, metastatic carcinoma and various forms of psoriasis, including psoriasis vulgaris and psoriasis eosinophilia; and myeloproliferative disorders and related disorders associated with activation JAK2 and myeloproliferative disorders and related disorders including, but are not limited, to myeloproliferative diseases such as, for example, polycythemia vera (PV), essential thrombocythemia (ET), myelofibrosis with myeloid metaplasia (MMM), also called chronic idiopathic myelofibrosis (CIMF), unclassified myeloproliferative disorders (uMPDs), hypereosinophilic syndrome (HES), and systemic mastocytosis (SM).

Lestaurtinib can be administered by any means that results in contact of the active agent with the agent's site of action in the body of the patient. Lestaurtinib can be administered by any conventional means available, either as individual therapeutic agents or in combination with other therapeutic agents. Lestaurtinib is preferably administered to a patient in need thereof in therapeutically effective amounts for the treatment of the diseases and disorders described herein.

Therapeutically effective amounts of lestaurtinib can be readily determined by an attending diagnostician by use of conventional techniques. The effective dose can vary depending upon a number of factors, including type and extent of progression of the disease or disorder, overall health of a particular patient, biological efficacy of the lestaurtinib, formulation of the lestaurtinib, and route of administration of the forms of lestaurtinib. Lestaurtinib can also be administered at lower dosage levels with gradual increases until the desired effect is achieved.

As used herein, the term “about,” as used herein, refers to a range of values from ±10% of a specified value. For example, the phrase “about 50 mg” includes ±10% of 50 or from 45 to 55 mg.

Typical dose ranges of lestaurtinib comprise from about 0.01 mg/kg to about 100 mg/kg of body weight per day or from about 0.01 mg/kg to 10 mg/kg of body weight per day. Daily doses for adult humans includes about 20, 25, 30, 35, 40, 45, 50, 55, 60, 65, 70, 75, 80, 90, 100, 120, 140, 160 and 200 mg and an equivalent dose for a human child. Lestaurtinib can be administered in one or more unit dose forms and can also be administered one to four times daily, including twice daily (BID). Unit dose ranges of lestaurtinib comprise from about 1 to about 400 mg administered one to four times a day, or from about 10 mg to about 200 mg BID, or 20-80 mg BID, or 60-100 mg BID or from about 4°, 6°, 80, or 100 mg BID.

Dosage of forms of lestaurtinib can also be in the form of liquids or suspensions in a concentration of between 15 to 25 mg/mL, 16 mg/mL or 25 mg/mL. The liquid or suspension dosage forms of lestaurtinib can include the equivalent of the doses (mg) described above. For example, dosages of lestaurtinib can include 1 to 5 mL of the 25 mg/mL solution, or 1, 1.2, 1.4, 1.6, 1.8, 2, 2.2, 2.4, 2.6, 2.8, 3, 3.2, 3.4, 3.6, 3.8, or 4 mL of the 25 mg/mL solution, wherein a 60 mg dose of lestaurtinib can be provided in 2.4 mL of solution, an 80 mg dose of lestaurtinib can be provided in 3.2 mL of solution and a 100 mg dose of lestaurtinib can be provided in 4 mL of solution. Additionally, a 20 mg dose of lestaurtinib can be provided with a 1.25 mL of a 16 mg/mL solution.

The daily dose of lestaurtinib can range from 1 mg to 5 mg/kg (normalization based on a mean body weight close to 65 kg). For example, a daily dose of a form of lestaurtinib is from about 1 to 3 mg/kg or from about 1.2 to 2.5 mg/kg, or about 1.2, 1.4, 1.6, 1.8, 2, 2.2, 2.4, 2.6, 2.8 or 3 mg/kg. In an alternate method of describing an effective dose, an oral unit dose of lestaurtinib is one that is necessary to achieve a blood serum level of about 0.05 to 20 μg/mL or from about 1 to 20 μg/mL in a patient.

Lestaurtinib can be formulated into pharmaceutical compositions by mixing the forms with one or more pharmaceutically acceptable excipients. It is meant to be understood that pharmaceutical compositions include any form of lestaurtinib or any combination thereof.

The term “pharmaceutically acceptable excipients,” as used herein, includes any and all solvents, dispersion media, coatings, antibacterial and antifungal agents, isotonic and absorption delaying agents and the like. The use of such media and agents for pharmaceutical active substances is well known in the art, such as in Remington: The Science and Practice of Pharmacy, 20^{th} ed.; Gennaro, A. R., Ed.; Lippincott Williams & Wilkins: Philadelphia, Pa., 2000. Except insofar as any conventional media or agent is incompatible with the active ingredient, its use in the therapeutic compositions is contemplated. Supplementary active ingredients can also be incorporated into the compositions.

Excipients for preparation of compositions comprising forms of lestaurtinib to be administered orally include, for example, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl celluose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water and mixtures thereof. Excipients for preparation of compositions comprising forms of lestaurtinib to be administered ophthalmically or orally include, for example, 1,3-butylene glycol, castor oil, corn oil, cottonseed oil, ethanol, fatty acid esters of sorbitan, germ oil, groundnut oil, glycerol, isopropanol, olive oil, polyethylene glycols, propylene glycol, sesame oil, water and mixtures thereof. Excipients for preparation of compositions comprising forms of lestaurtinib to be administered osmotically include, for example, chlorofluoro-hydrocarbons, ethanol, water and mixtures thereof. Excipients for preparation of compositions comprising forms of lestaurtinib to be administered parenterally include, for example, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water and mixtures thereof. Excipients for preparation of compositions comprising forms of lestaurtinib to be administered rectally or vaginally include, for example, cocoa butter, polyethylene glycol, wax and mixtures thereof.

Dosage forms of lestaurtinib and compositions comprising lestaurtinib depend upon the route of administration. Any route of administration is contemplated, including oral, mucosal (e.g. ocular, intranasal, pulmonary, gastric, intestinal, rectal, vaginal and uretheral) or parenteral (e.g. subcutaneous, intradermal, intramuscular, intravenous, or intraperitoneal.

Pharmaceutical compositions are most preferably administered orally, preferably in forms such as tablets, capsules, powders, pills, liquids/suspensions or gels/suspensions or emulsions, lyophillizates and all other different forms described in patents and applications mentioned herein, more preferably as tablets, capsules and liquids/suspensions or gels/suspensions. The administration vehicle can comprise one or more pharmaceutically acceptable carriers that are likely to ensure the solid state or crystalline form's stability (e.g. a suspension in oil).

Lestaurtinib can be formulated as a variety of pharmaceutical compositions and dosage forms, such as those described in U.S. Pat. Nos. 6,200,968 and 6,660,729 and PCT Publication No. 04/037928, each of which is incorporated herein by reference. In particular, the lestaurtinib can be formulated as microemulsions or dispersions.

In certain embodiments, compositions comprise lestaurtinib propylene glycol and a polyoxyethylene sorbitan fatty acid ester, examples of which include TWEEN® 20 (polyoxyethylene 20 sorbitan monolaurate), TWEEN® 40 (polyoxyethylene 20 sorbitan monopalmitate), and TWEEN® 80 (polyoxyethylene 20 sorbitan monooleate). In a particular embodiment, the lestaurtinib is present in a concentration of 25 mg/mL. In other embodiments, the ratio of the propylene glycol to the polyoxyethylene sorbitan fatty acid ester ranges from 50:50 to 80:20 or 50:50 or 80:20.

In other embodiments, compositions comprise lestaurtinib, a polyoxyl stearate and polyethylene glycol (“PEG”), examples of which include PEG of 300-800°, 400-3350 or 400-1500 Daltons or PEG-400, PEG-600, PEG-1000, PEG-1450, PEG-1500, PEG-400/PEG-1000, PEG-400/PEG-1450, PEG-600/PEG-1000 or PEG-600/PEG-1450.

In other still other embodiments, the polyoxyl stearate is polyoxyl 40 stearate (MYRJ 52®)). In particular embodiments, lestaurtinib is present in a concentration of 25 mg/mL. In other embodiments, the ratio of polyethylene glycol to the polyoxyl stearate ranges from 50:50 to 80:20 or ratios of 50:50 or 80:20. In certain embodiments, compositions comprise PEG-400, PEG-1000 and polyoxyl stearate in a ratio of 25:25:50 or PEG-400, PEG-1450 and polyoxyl stearate in a ratio of 25:25:50 or PEG-600, PEG-1000 and polyoxyl stearate in a ratio of 25:25:50 or PEG-600:PEG-1450:polyoxyl stearate in a ratio of 25:25:50. In other embodiments, the composition comprises PEG-400, PEG-1000 and polyoxyl stearate in a ratio of 40:40:20 or PEG-400, PEG-1450 and polyoxyl stearate in a ratio of 40:40:20 or PEG-600, PEG-1000 and polyoxyl stearate in a ratio of 40:40:20 or PEG-600, PEG-1450 and polyoxyl stearate in a ratio of 40:40:20.

In another embodiment of this invention, an the composition includes an antioxidant is in. The term “antioxidant,” as used herein, means a substance that retards deterioration by oxidation or inhibits reactions promoted by oxygen or peroxides. Antioxidants include, but are not limited to, ascorbic acid, fatty acid esters of ascorbic acid, butylated hydroxytoluene (BHT), propyl gallate, butylated hydroxyanisole, mixtures thereof and the like. In certain embodiments of this invention, microemulsions or solid solution compositions comprising lestaurtinib further comprise BHT, and in particular 0.02% w/w BHT.

Lestaurtinib and solvates thereof can be made by synthetic chemical processes, examples of which is shown hereinbelow. It is meant to be understood that the order of the steps in the processes may be varied, that reagents, solvents and reaction conditions may be substituted for those specifically mentioned, and that moieties succeptable to undesired reaction may be protected and deprotected, as necessary.

The following examples are presented to provide what is believed to be the most useful and readily understood description of procedures and conceptual aspects of this invention.

### PREPARATIVE EXAMPLE 1

Lestaurtinib and the methanolate thereof were prepared as described in U.S. Pat. No. 4,923,986.

### EXAMPLE 1

### Lestaurtinib Crystalline Form 1

A mixture of lestaurtinib methanolate in methanol and acetone was polish filtered. The filtrant was constant-volume distilled with addition of isopropyl acetate. When the boiling point of the solvent stabalized at 82° C., the mixture was cooled and filtered.

### EXAMPLE 2

### Crystalline Hydrated Lestaurtinib

A mixture of lestaurtinib (400 mg) in refluxing acetone (200 mL), in which the lestaurtinib was completely soluble, was treated with water until turbid, cooled, stored under darkness at ambient temperature for 3 days and filtered through a medium porosity sintered-glass funnel. The filtrant was washed with water and air-dried. Exposure of the product to relative humidity less than 40% provided crystalline lestaurtinib monohydrate. Exposure of the product to relative humidity of 40% or greater provided crystalline lestaurtinib trihydrate.

### EXAMPLE 2A

### Crystalline Hydrated Lestaurtinib

A mixture of lestaurtinib (1.2 g) in refluxing 1,3-dioxolane, in which the lestaurtinib was completely soluble (120 mL), was poured into water (600 mL), stored under darkness at ambient temperature for 6 days and filtered through a medium porosity sintered-glass funnel. The filtrant was washed with water (10 mL) and air-dried. Exposure of the product to relative humidity less than 40% provided crystalline lestaurtinib monohydrate. Exposure of the product to relative humidity of 40% or greater provided crystalline lestaurtinib trihydrate.

### EXAMPLE 3

### Crystalline Lestaurtinib Hemihydrate Hemiacetonitrileate

A solution of lestaurtinib (300 mg) in refluxing acetonitrile (150 mL), in which the lestaurtinib was completely soluble, was treated with water until turbid, cooled, stored under darkness at ambient temperature for 24 hours and filtered.

### EXAMPLE 4

### Amorphous Lestaurtinib

A mixture of lestaurtinib (1.6 g) in isopropanol (350 mL) and 1,3-dioxolane (50 mL) at 80° C., in which the lestaurtinib was completely soluble, was concentrated under vacuum. The concentrate was washed with isopropanol (10 mL) and air dried.

### EXAMPLE 4A

### Amorphous Lestaurtinib

A mixture of lestaurtinib in acetone, in which the lestaurtinib was completely soluble, was concentrated at 65° C. under vacuum. The concentrate was washed with isopropanol (10 mL) and air dried.

Additional ways to prepare amorphous lestaurtinib are shown in TABLE 1. Concentrations were conducted at about the temperature indicated in TABLE 1 at about 0.5 atm.

TABLE 3

| solvent | technique (bath temperature) |
|---|---|
| acetonitrile/reflux | concentration (stream of N₂ gas) |
| acetone | concentration (65° C.) |
| 1,3-dioxolane/isopropanol | concentration (80° C.) |
| 1,3-dioxolane/water | concentration (55° C.) |
| ethyl acetate | concentration (60° C.) |
| isopropanol | concentration (80° C.) |
| DMSO | antisolvent (water) |
| tetrahydrofuran | concentration (60° C.) |
| THF/methanol | antisolvent (hexanes) |

### EXAMPLE 5

### Crystalline Lestaurtinib Anhydrate

Hydrated crystalline lestaurtinib was heated between about 80° C. and 100° C. at about 760 mm Hg (1 atm) pressure. The product was stored in an environment having less than about 5% relative humidity.

### EXAMPLE 6

### Lestaurtinib Crystalline Form 1

A mixture of EXAMPLE 2, EXAMPLE 2A, EXAMPLE 4, EXAMPLE 4A or a mixture thereof in ethanol, in which the example, or the mixture thereof, was partially soluble, was allowed to stand, with or without stirring, until Lestaurtinib Crystalline Form 1 formed.

### EXAMPLE 7

### Crystalline Lestaurtinib Hemihydrate Hemitetrahydrofuranate

A mixture of lestaurtinib in refluxing THF, in which the lestaurtinib was completely soluble, was treated with water until turbid, cooled, stored under darkness at ambient temperature for 24 hours and filtered.

It is meant to be understood that peak heights in a PXRD spectrum may vary and will be dependent on variables such as the temperature, size of crystal size or morphology, sample preparation, or sample height in the analysis well of the Scintag×2 Diffraction Pattern System.

It is also meant to be understood that peak positions may vary when measured with different radiation sources. For example, Cu—Kα₁, Mo—Kα, Co—Kα and Fe—Kα radiation, having wavelengths of 1.54060 Å, 0.7107 Å, 1.7902 Å and 1.9373 Å, respectively, may provide peak positions that differ from those measured with Cu—Kα radiation.

The term “about” preceding a series of peak positions is meant to include all of the peak positions of the group which it precedes.

The term “about” preceding a series of peak positions means that all of the peaks of the group which it precedes are reported in terms of angular positions with a variability of ±0.1°.

For example, the phrase about 6.8°, 8.5°, 9.7°, 12.0°, 13.2°, 14.2°, 14.7°, 15.0°, 15.5°, 16.9°, 17.5°, 17.9°, 19.3°, 20.0°, 20.4°, 25.1°, 25.6°, 25.8°, 26.3° or 26.6° means about 6.8°, about 8.5°, about 9.7°, about 12.0°, about 13.2°, about 14.2°, about 14.7°, about 15.0°, about 15.5°, about 16.9°, about 17.5°, about 17.9°, about 19.3°, about 20.0°, about 20.4°, about 25.1°, about 25.6°, about 25.8°, about 26.3° or about 26.6° and also 6.80±0.1°, 8.5°±0.1°, 9.7°±0.1°, 12.0°±0.1°, 13.2°±0.1°, 14.2°±0.1°, 14.7°±0.1°, 15.0°±0.1°, 15.5°±0.1°, 16.9°±0.1°, 17.5°±0.1°, 17.9°±0.1°, 19.3°±0.1°, 20.0°±0.1°, 20.4°±0.1°, 25.1°±0.1°, 25.6°±0.1°, 25.8°±0.1°, 26.3°±0.1° or 26.6°±0.1°.

As those skilled in the art will appreciate, numerous modifications and variations of the present invention are possible in view of the above teachings. It is therefore understood that within the scope of the appended claims, the invention can be practiced otherwise than as specifically described herein, and the scope of the invention is intended to encompass all such variations.

We claim:

1. Lestaurtinib Crystalline Form 1 characterized, when measured at about 25° C. with Cu—Kα radiation, by a powder diffraction pattern with at least three peaks having respective 2θ values of about 6.8°, 8.5°, 9.7°, 12.0°, 13.2°, 14.2°, 14.7°, 15.0°, 15.5°, 16.9°, 17.5°, 17.9°, 19.3°, 20.0°, 20.4°, 25.1°, 25.6°, 25.8°, 26.3° or 26.6°.

2. Lestaurtinib Crystalline Form 1 characterized, in the triclinic crystal system and P1 space group, when measured at about −100° C. with Mo—Kα radiation, by lattice parameters a, b and c of 11.235 Å±0.002 Å, 13.317 Å±0.004 Å, 7.095 Å±0.003 Å, respectively, and α, β and γ of 92.33°±0.03°, 107.78°±0.02° and 100.95°±0.02°, respectively.

3. A composition comprising Lestaurtinib Crystalline Form 1 and an excipient.

4. A method of treating a patient having acute myeloid leukemia comprising administering thereto a therapeutically acceptable amount of Lestaurtinib Crystalline Form 1.

5. A method of treating a patient having chronic myeloid leukemia comprising administering thereto a therapeutically acceptable amount of Lestaurtinib Crystalline Form 1.

6. A method of treating a patient having acute lymphocytic leukemia comprising administering thereto a therapeutically acceptable amount of Lestaurtinib Crystalline Form 1.

7. A method of treating a patient having chronic lymphocytic leukemia comprising administering thereto a therapeutically acceptable amount of Lestaurtinib Crystalline Form 1.

8. A process for making Lestaurtinib Crystalline Form 1 comprising providing a mixture comprising the lestaurtinib, or the solvate thereof, and solvent, wherein the lestaurtinib, or the solvate thereof, is completely dissolved in the solvent and causing Lestaurtinib Crystalline Form 1 to exist in the mixture, the Lestaurtinib Crystalline Form 1, when isolated and measured at about −100° C. with Mo—Kα radiation, characterized the triclinic crystal system and P1 space group by lattice parameters a, b and c of 11.235 Å±0.002 Å, 13.317 Å±0.004 Å, 7.095 Å±0.003 Å, respectively, and α, β and γ of 92.33°±0.03°, 107.78°±0.02° and 100.95°±0.02°, respectively.

9. The process of claim 8 further comprising isolating the Lestaurtinib Crystalline Form 1.

10. In a process for making Lestaurtinib Crystalline Form 1 comprising treatment of a carboxyl-protected intermediate in said process with a reducing agent and subsequently crystallizing or recrystallizing said lestaurtinib to said Lestaurtinib Crystalline Form 1, said process comprising direct crystallization of Lestaurtinib Crystalline Form 1 from a solid, semisolid or syrup having therewith one or more than one solvent from said carboxylic acid-protected intermediate reduction.
